(19) United States
(12) Patent Application Publication
(10) Pub. No.: US 2007/0116725 A1
(43) Pub. Date: May 24, 2007

(54) Live Attenuated Samonella Strains for Producing Monovalent or Multivalent Vaccines

(76) Inventors: Ion R. Vladoianu, Cologny (CH); Jose A. Berdoz, Chernex (CH)

Correspondence Address: MINTZ, LEVIN, COHN, FERRIS, GLOVSKY;AND POPEO, P.C., ONE FINANCIAL CENTER, BOSTON, MA, 02111, US

(21) Appl. No.: 11/567,043

(22) Filed: Dec. 5, 2006

Related U.S. Application Data
(63) Continuation of application No. 10/011,960, filed on Nov. 5, 2001.
(60) Provisional application No. 60/327,472, filed on Oct. 4, 2001.

(51) Int. Cl. A61K 39/112; C12N 1/21; C12N 15/74

(52) U.S. Cl. 424/258.1; 435/472; 435/252.3

(57) ABSTRACT

Disclosed herein methods for producing live attenuated Salmonella typhi, Salmonella paratyphi A and B and other Salmonella mutants which can be used in vaccines to prevent diseases caused by Salmonella infection. These mutants can also be used to prevent or treat diseases caused by other bacterial strains, by viral and parasitic pathogens and by tumor cells.

### RELATED APPLICATIONS

This application is a continuation of U.S. patent application Ser. No. 10/011,960, filed Nov. 5, 2001 which claims the benefit of application Ser. No. 60/327,472, filed Oct. 4, 2001. The contents of these applications are incorporated herein by reference in their entirety.

### FIELD OF THE INVENTION

The present invention relates to the preparation of live attenuated Salmonella typhi, Salmonella paratyphi A and B and of other live attenuated Salmonella mutants which can be used in methods to prevent diseases caused by Salmonella infection. These mutants can also be used to prevent or treat diseases caused by other bacterial strains, by viral and parasitic pathogens and by tumor cells.

### BACKGROUND OF THE INVENTION

Enteric diseases caused by Salmonella bacteria, Salmonellosis, is an important global health problem, particularly in the developing world (Ivanoff et al., ANN. MED. INT., 149: 340-350 (1998); Pang et al., TRENDS MICROBIOL. 6: 131-133 (1998)). Moreover, the incidence of enteric fevers caused by multi-drug-resistant Salmonella typhi (S. typhi) and Salmonella paratyphi (S. paratyphi) is continuously increasing all over the world (Akinyemi et al., ZEITSCHRIFT FÜR NATURFORSCHUNG, SECTION C, 55: 489-493 (2000); Chandel et al., EMERG. INFECT. DIS., 6: 420-421 (2000)). These observations underscore the importance of vaccination as an alternate medical route to control Salmonella-related diseases (Hampton et al., EMERG. INFECT. DIS., 4: 317-320 (1998); Mermin et al., ARCH. INT. MED., 158: 633-638 (1998)).

In spite of the significant efficiency of the anti-typhoid vaccines currently marketed, including the killed vaccines, the live attenuated Ty 21a vaccine (Vivotif®)), the Vi-polysaccharide vaccine (Typhim®) and of the live attenuated Salmonella strains currently tested in clinical trials, there is a great demand for other live attenuated Salmonella strains with improved properties. Indeed, each of these vaccines is associated with at least one drawback of sufficient concern that there is interest in the development of additional candidates S. typhi vaccine strains. Moreover, no vaccine is available against the paratyphoid fevers and effective anti-paratyphoid vaccines are urgently needed to protect travelers from developed countries that visit endemic regions, to prevent disease outbreaks in industrialized countries, and to tackle endemicity in developing countries.

The development of a novel typhoid and paratyphoid A and B (TAB) vaccine based on live attenuated strains to prevent both typhoid and paratyphoid A and B fevers is highly desirable. Indeed, such combined vaccines would reduce the number of immunizations and the associated cost of the vaccination programs. Accordingly, there is still a need in the art for efficacious, low risk, and cost effective vaccines, administered preferably in a single dose via the oral route, to protect against typhoid and paratyphoid fevers.

### SUMMARY OF THE INVENTION

The present invention provides live, attenuated bacterial mutants that are derived from pathogenic strains. These mutants have two of the following characteristics: (i) resistance or dependence to an antibiotic; or (ii) resistance to a virulent bacteriophage. The bacteriophage binds to an antigen that is one of the main virulence factor of the pathogenic strain.

The present invention also provides live, attenuated bacterial mutants that are derived from pathogenic enteric strains. These mutants have at least two of the following characteristics:

(i) resistance or dependence to an antibiotic; (ii) resistance to a virulent bacteriophage; or (iii) resistance to bile salts.

An object of the present invention provides attenuated strains of Salmonella that can be used as live vaccines and as live vectors for foreign antigens and for foreign DNA. These live attenuated Salmonella strains constitute an invaluable tool for the preparation of new vaccines not only against typhoid and paratyphoid fevers, but also against diseases caused by pathogens of viral, parasitic, and bacterial origin and to target selectively tumor cells.

Another object of the present invention provides a method to achieve the attenuation of virulent wild-type Salmonella and the selection of the resulting live attenuated Salmonella. The Salmonella mutant strains resulting from this method are: (i) resistant or dependent to an antibiotic; (ii) resistant to a virulent bacteriophage; (iii) resistant to a bile salts preparation. In particular, the Salmonella mutant strains are: (i) resistant or dependent to streptomycin; (ii) resistant to the Felix O bacteriophage or to any other virulent bacteriophage whose receptor or co-receptor is located on the lipopolysaccharide (LPS); (iii) resistant to cholic or deoxycholic acid or to both cholic and deoxycholic acids.

Still another object of the present invention provides live attenuated Salmonella which is substantially incapable of reverting to full virulence in the amount of mutants contained in the pharmaceutically effective dosage. The Salmonella mutant strains contain at least two independent mutations and residual virulence of the mutants is evaluted by both in vitro and in vivo assays.

Yet another object of the present invention provides live attenuated Salmonella which express an heat-stable anchoring of the Vi antigen in the bacterial membrane. In particular, some of the S. typhi mutants express a Vi antigen that is not released from the bacterial membrane after heating 10 nm at 100° C. (boiling water).

An additional object of the present invention provides mucosal vaccines against diseases caused by Salmonella, like typhoid and paratyphoid fevers. Vaccines can be prepared by combining one or more live attenuated Salmonella strains with a pharmaceutically acceptable diluent or carrier.

A further object of the present invention provides attenuated Salmonella which can be used as live vectors for foreign genes cloned from other pathogens, that will be expressed into proteins, and will raise protective immune responses against the pathogens from which they are derived.

A still further object of the present invention provides attenuated Salmonella strains which can be used as live vectors to deliver DNA-mediated vaccines.

These and other objects of the present invention will be apparent from the detailed description of the invention provided below.

### BRIEF DESCRIPTION OF THE DRAWINGS

The present invention will be further understood from the following description with reference to the figures, in which:

FIG. 1 is a flow diagram detailing the procedure used to obtain and select the live attenuated mutant strains from S. typhi, S. paratyphi A, and S. paratyphi B. The nomenclature of the mutants is included.

FIG. 2 is a table that compares select characteristics of the wild-type strains and of the live attenuated streptomycin-resistant mutant strains of S. typhi, S. paratyphi A, and S. paratyphi B. Specifically, the table summarizes the growth properties of these Salmonella strains on media containing different concentrations of bile salts, their sensitivity to streptomycin and to Felix 0 bacteriophage. Also shown, is the slide agglutination of these Salmonella strains, heated for 10 minutes at 100° C. (boiling water) or not heated, with antisera directed against O-antigen, Vi-antigen and H-antigen.

FIG. 3 is a table that compares select characteristics of some representative live attenuated S. typhi mutant strains that are streptomycin-resistant or streptomycin-dependent, resistant or sensitive to Felix O bacteriophage. Specifically, the table summarizes the growth properties of these Salmonella strains on media containing different concentrations of bile salts, their sensitivity to streptomycin and to Felix O bacteriophage. Also shown, is the slide agglutination of these Salmonella strains, heated for 10 minutes at 100° C. or not heated, with antisera directed against O-antigen, Vi-antigen and H-antigen. Further shown, is the frequency of reversion to bile salts resistance of some of these Salmonella strains.

FIG. 4 is a table that compares select characteristics of the wild-type S. typhi and of some representative live attenuated S. typhi mutant strains that are streptomycin-resistant or streptomycin-dependent, resistant or sensitive to Felix O bacteriophage. Specifically, the effect of osmolarity on the expression of O-antigen, Vi-antigen, and H-antigen is assessed by slide agglutination with specific antisera.

FIG. 5 is a table that compares select characteristics of some representative live attenuated S. paratyphi A mutant strains that are streptomycin-resistant, resistant or sensitive to Felix O bacteriophage. Specifically, the table summarizes the growth properties of these Salmonella strains on media containing different concentrations of bile salts, their sensitivity to streptomycin and to Felix O bacteriophage. Also shown, is the slide agglutination of these Salmonella strains with antisera directed against O-antigen and H-antigen. Further shown, is the frequency of reversion to bile salts resistance of some of these Salmonella strains.

FIG. 6 is a table that compares select characteristics of some representative live attenuated S. paratyphi B mutant strains that are streptomycin-resistant, resistant or sensitive to Felix O bacteriophage. Specifically, the table summarizes the growth properties of these Salmonella strains on media containing different concentrations of bile salts, their sensitivity to streptomycin and to Felix O bacteriophage. Also shown, is the slide agglutination of these Salmonella strains with antisera directed against O-antigen and H-antigen. Further shown, is the frequency of reversion to bile salts resistance of some of these Salmonella strains.

FIG. 7 is a table that compares the sensitivity of some representative live attenuated S. typhi, S. paratyphi A and S. paratyphi B mutant strains to bile salts and to Triton X-100, used to lyse the THP-1 cells in the monocyte-derived macrophages survival assay (FIG. 8).

FIG. 8 is a table that compares the survival after 5 hours and 24 hours of the wild-type strains and of some representative live attenuated mutant strains of S. typhi, S. paratyphi A and S. paratyphi B in the monocyte-derived macrophage cell line THP-1. Also shown, is the resistance of these Salmonella strains to the bactericidal effect of human serum.

FIG. 9 is a table that compares the survival of the wild-type strain S. typhi Ty in monocyte-derived macrophages prepared from a human volunteer before and after vaccination with the S. typhi mutant strain Ty V2.

### DETAILED DESCRIPTION OF THE INVENTION

I. Prior Art Vaccines Against Typhoid and Paratyphoid Fevers

As described below, several vaccines have been developed against typhoid and paratyphoid fevers. However, currently available vaccines suffer from a number of disadvantages, such as the induction of intense local and systemic reactions, partial protection, induction of vaccinemias, or the need for multiple doses.

A. Killed Vaccines Against Typhoid and Paratyphoid Fevers

The heat-inactivated, phenol-preserved whole cell S. typhi vaccine available one hundred years ago and administered via the parenteral route was shown to confer moderate levels of protection (51 to 67% efficacy) that endured for up to seven years (Ashcroft et al., AM. J. HYG., 79: 196-206 (1964); Ashcroft et al., LANCET, 2: 1056-1060 (1967)). Although this vaccine was essentially safe, frequent local and systemic adverse reactions were observed. However, this vaccine is still among the three licensed vaccines against typhoid with the Vivotif® (Ty 21a live attenuated S. typhi strain) and the Typhim® (Vi antigen).

Killed TAB vaccine, administered either by parenteral injection or in oral form, to prevent typhoid and paratyphoid A and B fevers, was of limited efficacy in both adults and children. Humoral protective immune response was good for S. paratyphi A, intermediate for S. typhi and poor for S. paratyphi B (Dimache et al, ARCH. ROUM. PATH. EXP. MICROBIOL., 26: 747-759 (1967); Vladoianu et al., BULL. WHO, 32: 37-45 (1965)). In addition, parenteral administration of this vaccine induced intense local and systemic reactions that make general acceptance of the TAB vaccine difficult. However, the orally administered killed TAB vaccine was reported to be well tolerated. Because of these limitations, the TAB vaccine is not presently licensed for use in the United States and in many other countries (WHO Expert Committee Report on Biol. Stand., WHO TECH. REP. SER., 361: 65 (1967); Arya et al., VACCINE, 13: 1727-1728 (1995)). Actually, no other vaccine replaces the killed TAB vaccine.

B. Parenteral Polysaccharide Vaccines Against Typhoid and Paratyphoid Fevers

The capsular Vi polysaccharide of S. typhi, also present on S. paratyphi C, as well as on a few strains of S. dublin and Citrobacter freundii but not present on S. paratyphi A and B is both an important virulence factor and a protective antigen (Felix et al., LANCET, 227: 186-191 (1934); Robbins et al., J. INFECT. DIS., 150: 436-449 (1984)). Purified Vi antigen, administered in one dose as an intramuscular or deep subcutaneous injection was shown to be well-tolerated and to induce protective immunity in 64 to 90% of the volunteers for at least three years (Acharya et al., N. ENGL. J. MED., 317: 1101-1104 (1987); Klugman et al., VACCINE, 14: 435-438 (1996); Tacket et al., VACCINE, 6: 307-308 (1988)). Efficacy of these trials is quite variable and depends on the proportion of infants enrolled in the study and their age. Indeed, like most polysaccharide vaccines, the Vi vaccine does not induce either protective levels of antibodies in young children or a booster response (Landy et al., AM. J. HYG., 60: 52-62 (1954); Keitel et al, VACCINE, 12: 195-199 (1994)). To overcome the limitations of the age-related and T-cell independent immunogenicity of the vaccine, Vi was bound to Pseudomonas aeruginosa exotoxin A (EPA) and tested in a large group of two to five years old children for whom no effective vaccine was previously available. In this trial, the efficacy of the Vi-conjugate vaccine was 91% with no serious adverse reactions (Lin et al., N. ENGL. J. MED., 344: 1263-1269 (2001)). However, both the Vi and the Vi-EPA vaccines do not protect against S. typhi strains that lack the Vi antigen.

Similarly, S. paratyphi A O-specific polysaccharide was bound to tetanus toxoid (SPA-TT) and administered parenterally in adults, teenagers and children (Konadu et al., INFECT. IMMUN., 68: 1529-1534 (2000)). None of the vaccinees experienced significant side effects and in all of them an increase of anti-LPS antibody titers (IgG and IgM) was observed. A gradual decline in the IgG anti-LPS levels at 180 days was observed to be age related. In addition, the SPA-TT antigen did not elicit a booster response in the children in contrast to the booster response elicited in the same age group by the Vi-EPA vaccine. It is presently not predictable whether this strategy may be improved, for example by coupling the O-specific polysaccharide to another carrier molecule.

C. Live Attenuated Vaccines Against Typhoid Fever

The first live attenuated strains tested were streptomycin-dependent (SmD) strains of S. typhi (Reitman, J. INFECT. DIS., 117: 101-107 (1967); Cvjetanovic et al., BULL. WHO, 42: 499-507 (1970)). When administered orally, these strains were shown to be well tolerated and conferred 80% protection against experimental challenge (Dupont et al., ANTIMICROB. AGENTS CHEMOTHER., 10: 236-239 (1970)). However, when a lyophilized vaccine was reconstituted and administered, it lost its efficacy (Levine et al., J. INFECT. DIS., 133: 424-429 (1976)). Although the reason for this discrepancy is unknown, further studies with the SmD strains were discontinued.

The most extensively studied live attenuated strain derived from S. typhi is the Ty 21a (Vivotif®) oral vaccine marketed by Berna, Switzerland. Ty 21a has been obtained by chemical mutagenesis (Germanier et al, J. INFECT. DIS., 131: 553-558 (1975)). Whereas Ty 21a has proven to be remarkably well-tolerated by both adults and children, formulation of the vaccine and the number of doses markedly influence the level of protection that can be achieved (Black et al., VACCINE, 8: 81-84 (1990); Levine et al., LANCET, 1: 1049-1052 (1987)). Three or four doses of lyophilized Ty 21a vaccine in enteric-coated, acid-resistant, capsules constitute the commercial product that confers a protective efficacy ranging from 67% to 96% depending on the country where the clinical study was conducted (Wahdan et al., J. INFECT. DIS., 145: 292-295 (1982); Levine et al., LANCET, 1: 1049-1052 (1987)). Subsequently, a liquid formulation was shown to provide superior protection than enteric-coated capsules and administration of three doses elicited 77% protection over the same period (Levine et al., VACCINE, 17 (Suppl. 2): 22-27 (1999)). However, the Vivotif® has several drawbacks including its moderate immunogenicity, the need to administer at least three doses and the fact that Ty 21a is Vi-negative and does not stimulate the immune system to produce anti-Vi antibodies. One attempt to increase the immunogenicity of the Ty 21a strain was to restore the expression of the Vi antigen (Cryz et al., INFECT. IMMUN., 57: 3863-3868 (1989)). The resulting Vi-positive Ty21a administered to volunteers was well tolerated but none of them developed anti-Vi antibodies whereas most of them were still able to produce anti-O (anti-LPS) antibodies (Tacket et al., J. INFECT. DIS., 163: 901-904 (1991)).

Many other live attenuated S. typhi strains have been engineered in such a way that they are more immunogenic than the Ty 21a and may elicit protective immunity after the administration of a single dose. In this process, genes encoding various biochemical pathways, global regulatory factors, heat shock proteins, and virulence factors have been inactivated (Levine et al., BEHRING INST. MITT., 98: 120-123 (1997). Some of these mutants have been tested in clinical trials and were shown to be poorly immunogenic, like 541Ty (aroA and purA mutant) and 543 Ty (aroA, purA, Vi-mutant) (Levine et al., J. CLIN. INVEST., 79: 888-902 (1987)), or insufficiently attenuated like EX462 (gale, Vi mutant) and X3927 (cya, crp mutant) (Hone et al., INFECT. IMMUN., 56: 1326-1333 (1988); Curtiss et al., DEV. BIOL. STAND., 82: 23-33 (1994)). However, several other mutants were attenuated enough to avoid most of the side effects and typhoid-like symptoms but immunogenic enough to be considered as possible vaccine candidates. Among these candidates, Ty800 (PhoP/PhoQ mutant) (Hohmann et al., J. INFECT. DIS., 173: 1408-1414 (1996)), X4073 (cya, crp, cdt mutant) (Tacket et al., INFECT. IMMUN., 65: 3381-3385 (1997)), CVD908 (aroC, aroD mutant) (Sztein et al., J. INFECT. DIS., 170: 1508-1517 (1994)), and CVD908-htrA (aroC, aroD, htrA mutant) (Tacket et al., INFECT. IMMUN., 65: 452-456 (1997); Tacket et al., INFECT. IMMUN., 68: 1196-1201 (2000)) generated potent immune response and protection but each was associated with at least one drawback. Indeed, these four strains did not generate anti-Vi antibodies known to play an important role in the protective immune response and provoked in some vaccinees mild diarrhea or vaccinemias.

Although encouraging, it was thought that these data could still be improved by selection of other live attenuated mutants with more appropriate properties. CVD915 (guaB-A mutant), CVD916 (guaB-A, Vi-constitutive mutant) derived from CVD915 and CVD909 (aroC, aroD, htrA, Vi-constitutive mutant) derived from CVD908-htrA were tested in mice for their capacity to induce a stronger anti-Vi response (Pasetti et al., CLIN. IMMUNOL., 92: 76-89 (1999); Wang et al., INFECT. IMMUN., 68: 4647-4652 (2000)). The data showed that the immune response against the Vi antigen was enhanced without interfering with the immune response against the LPS and the flagellar H antigen. It remains to be determined whether CVD916 and CVD909 will elicit a stronger production of anti-Vi antibodies as compared with CVD 915 in human volunteers.

II. Prior Art Vaccines Against Heterologous Pathogens

Attenuated strains of Salmonella have been shown to be efficient tools in inducing protective immunity against Salmonellosis. In addition, their potential as vehicles for the expression and delivery of heterologous antigens to the immune system has been illustrated both in animal models and in human volunteers with a variety of antigens from human and animal pathogens, including virulence antigens from bacteria, viruses, and protozoans (INTRACELLULAR BACTERIAL VACCINE VECTORS, Paterson ed., Wiley-Liss (1999)). Expression of these heterologous antigens has resulted in the induction of both humoral and cell-mediated immune responses to purified recombinant antigen and, in some instances, to the organism from which the antigen was cloned. Indeed, mammalian, viral and protozoan antigens expressed in prokaryotes may be sensitive to the bacterial proteases, form inclusion bodies or lose tertiary conformation and, consequently, do not elicit a protective immune response. However, when expressed in eukaryotic cells, such antigens may recover native antigenic conformation and elicit protection. This can be achieved when the gene encoding the foreign antigen is cloned into an eukaryotic expression vector and delivered to the mammalian cells by the Salmonella (Darji et al., CELL, 91: 765-775 (1997)). These studies demonstrated that Salmonella vectors possess the capacity to deliver nucleic acid vaccines. The potential of Salmonella vectors to induce antitumor immunity has also been suggested by several studies (Medina et al., EUR. J. IMMUNOL., 29: 693-699 (1999); Zheng et al., ONCOL. RES., 12: 127-135 (2000)).

However, the restricted number of live attenuated Salmonella strains presently available, that are well-tolerated and immunogenic in volunteers, hinder the development of promising vaccines. One example is the development of a vaccine against gastritis caused by Helicobacter pylori (H. pylori) which was based on the ureases A and B expressed in the live attenuated Salmonella Ty800 and which did not elicit any specific humoral immune response (DiPetrillo et al., VACCINE, 18: 449-459 (2000)). This strain was thought to be inappropriately attenuated for efficient presentation of heterologous antigens to the human immune system. Another report comparing mechanisms of immunity induced by different attenuated Salmonella in mice also supported the hypothesis that some strains are more appropriate than others as vectors (VanCott et al., NATURE MED., 11: 1247-1252 (1998)).

The future for Salmonella vectors looks very promising and will have a significant impact on mucosal vaccines development and tumor targetting. However, these developments will be highly dependent on the availability of novel live attenuated Salmonella.

III. The Live Attenuated Salmonella Mutants of the Present Invention

The present invention provides live, attenuated Salmonella mutant strains for use, inter alia, as live vaccines against Salmonella-related diseases, and as live vaccines against other diseases when used as vectors to deliver foreign antigens or foreign DNAs. A “mutant strain”, as used herein, is a strain that contains at least two mutations in the DNA sequence as compared with the corresponding parental strain. Mutations include e.g., base changes, deletions, insertions, inversions, translocations or duplications. A “microorganism”, as used herein, is a bacteria, a virus, a protozoa, or a fungi. As used herein, a “foreign antigen” or a “foreign DNA” means an antigen or a DNA foreign to Salmonella.

Also, in the present invention, “attenuated Salmonella” mutants are provided, wherein said mutants are less virulent than wild-type strains, yet able to induce either humoral or cellular immunity or both.

Further, in the present invention, “attenuated Salmonella” mutants are provided, wherein said mutants are substantially incapable of reverting to full virulence when administered at a pharmaceutically effective dose. As mentioned above, the attenuated Salmonella mutants of the present invention have at least two mutations in the DNA sequence as compared with the corresponding parental strain. Since the rate of reversion to wild-type for each mutation is very low, the probability of reversion of two or more mutations in one mutant is significantly lower than the number of mutants administered at a pharmaceutically effective dose.

Still further, in the present invention, “attenuated Salmonella” mutants are provided, wherein said mutants are obtained and selected for their resistance or dependence to (i) streptomycin, for their resistance to (ii) a bacteriophage, such as Felix 0, for their resistance to (iii) bile salts. The particular bacteriophage employed in the present invention is not critical thereto. Examples of such bacteriophages include virulent bacteriophages that induce lysis of the wild-type Salmonella and that have their receptors or co-receptors in the lipopolysaccharide (LPS) which is a bactrial virulence factor.

In addition, in the present invention, “attenuated Salmonella” mutants that express the Vi antigen are provided, wherein some of the said mutants express an heat stable anchoring of the Vi antigen into the bacterial membrane as shown with some of the mutants derived from S. typhi. In the present invention, the live attenuated Salmonella mutant strains were obtained by selection of naturally occurring genetic mutations but without employing mutagens, plasmids or transposons.

Virulence of the mutants is evaluated in vitro by the survival in the monocyte-derived macrophages assay and by the bactericidal effect of normal human serum and in vivo in an appropriate host.

A vaccine, as used herein, is a preparation including materials in combination with a suitable carrier that generate a desired biological response, e.g., an immune response when administered in a sensible host. The vaccine may 2107, in which case it is usually administered mucosally, including orally, or at least one killed organism or component thereof, in which case it is usually administered parenterally. The bacterial cells used for the vaccine of the invention are preferably administered alive via the mucosa.

A. Vaccines Against Typhoid and Paratyphoid Fevers

The particular S. typhi, S. paratyphi A, and S. paratyphi B employed as starting materials in the present invention are not critical thereto. In the examples herein, the S. typhi mutants were constructed from the virulent wild-type S. typhi strain Ty2 whereas the S. paratyphi A and B mutants were constructed from virulent wild-types strains isolated in Indonesia by blood culture from patients with paratyphoid fever. S. typhi Ty2 is a reference strain that can be obtained from a variety of sources, such as the American Type Culture Collection (ATCC), the Institut Pasteur (France) and the Imperial College (England).

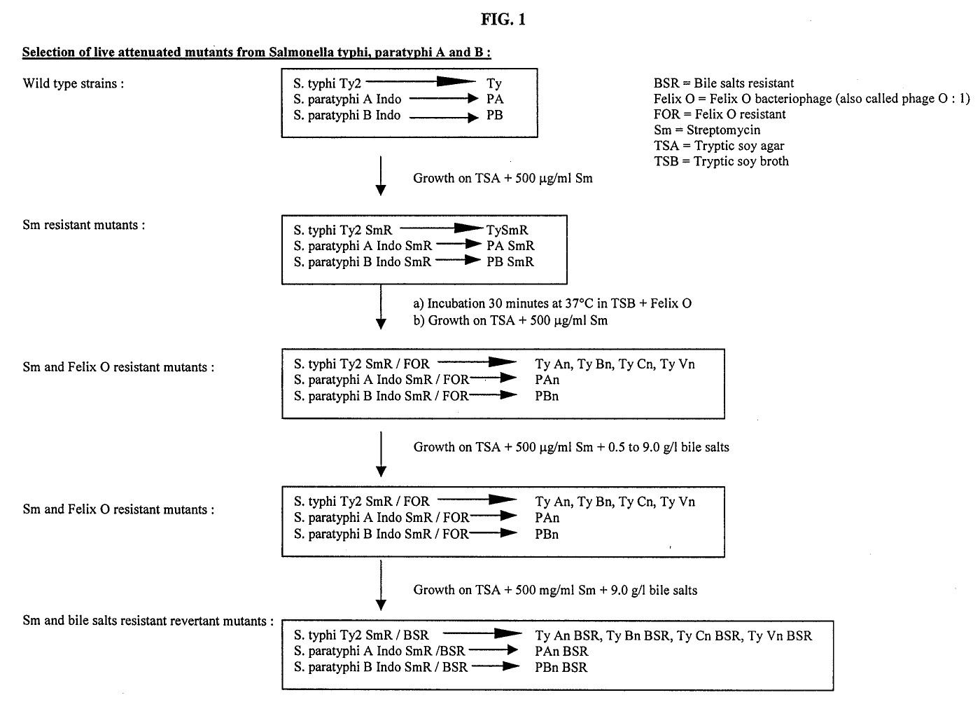

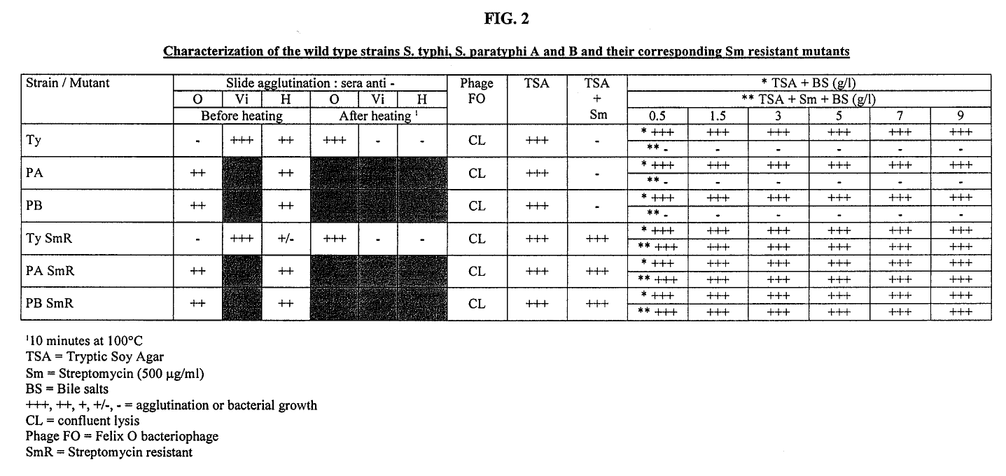

S. typhi (S. typhi Ty2), S. paratyphi A (S. paratyphi A Indo), and S. paratyphi B (S. paratyphi B Indo), respectively called Ty, PA, and PB (FIG. 1) were grown on Tryptic soy agar (TSA, Difco) or in Tryptic soy broth (TSB, Difco) and characterized by serological identification (slide agglutination with specific antisera, Sanofi Pasteur, FIG. 2), by biochemical properties (ID32E strip, bioMerieux), by susceptibility to antibiotics (ATB Vet strip, bioMerieux), and by susceptibility to the Felix O bacteriophage (also called phage O: 1, Sanofi Pasteur). Characterization of the Ty wild-type strain and derived mutants were performed with sera anti-O (O: 9), anti-Vi and anti-H(H: d). PA wild-type strain and derived mutants were characterized with sera anti-O (O: 1,2) and anti-H(H: a). PB wild-type strain and derived mutants were characterized with sera anti-O (O: 4,5) and anti-H(H: b). The sera are from Sanofi Pasteur. Agglutinations were assessed by visual examination and reported in FIGS. 2, 3, 4, 5, and 6 as +++, ++, +, +/−or −. Phage sensitivity was tested by application of drops of phage (Sanofi Pasteur) to TSA or TSA supplemented with 500 μg/ml of streptomycin inoculated with a bacterial suspension. Lysis was observed after incubation at 37° C. for at least 6 h. Identity of the wild-type strains was confirmed and no particular resistance to antibiotics commonly used to treat typhoid and paratyphoid fevers were observed.

Selection of streptomycin-resistant mutants of Ty, PA, and PB was made in a single step by plating the wild-type strains on TSA supplemented with a high concentration of streptomycin sulfate (500 μg/ml, Sigma). These mutants were called Ty SmR, PA SmR, and PB SmR (FIG. 1) and characterized by slide agglutination with specific antisera and by susceptibility to the Felix O bacteriophage. The SmR mutants did not differ significantly from their parent strains for the expression of the common surface antigens, for their sensibility to the Felix O bacteriophage and for their growth on TSA supplemented with bile salts No 3 (Difco), hereunder named bile salts, up to a concentration of 9 g/l (FIG. 2).

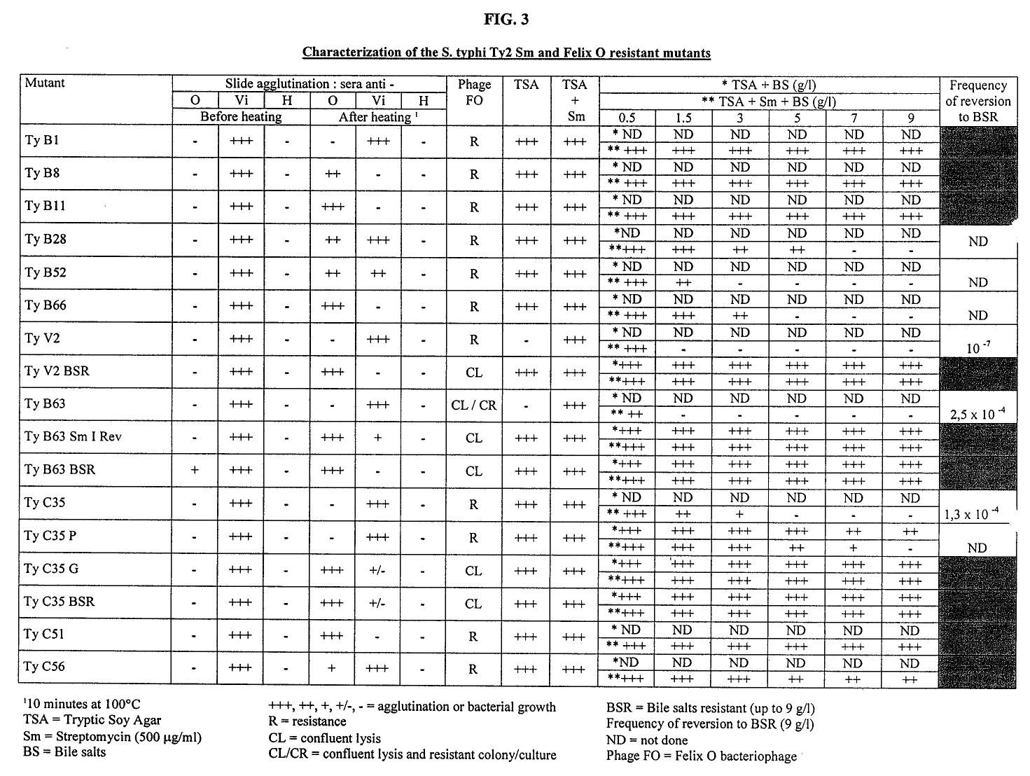

Selection of Felix O bacteriophage-resistant (FOR) mutants of Ty SmR, PA SmR, and PB SmR mutants was made by incubation of the SmR mutants in TSB with a 10-fold excess of phage O: 1 at 37° C. for 30 min. The resulting FOR mutants were then grown on TSA supplemented with 500 μg/ml of streptomycin. These mutants were called Ty An, Ty Bn, Ty Cn, Ty Vn, PAn, and PBn (where n=1, 2, 3, etc. FIG. 1) and characterized by slide agglutination with specific antisera and by susceptibility to the Felix O bacteriophage (FIGS. 3, 5, and 6).

The SmR-FOR mutants derived from Ty were shown to be composed of two clearly distinct classes: those which are Vi-positive after heating (10 minutes at 100° C.) and those which are Vi-negative after heating. In addition to the Ty SmR-FOR mutants, a few mutants were dependent to streptomycin (SmD) and resistant or partially resistant to the Felix O bacteriophage, like Ty V2 and Ty B63 that are Ty SmD-FOR mutants. Some of the SmR-FOR mutant strains grew well on media containing 9 g/l of bile salts. Others, including the SmD-FOR Ty V2 and Ty B63 mutants, were more sensitive to the presence of bile salts in the growth medium and stopped growing at bile salt concentrations lower than 9 g/l (FIG. 3). Although the mutants sensitive to bile salts may not display optimal characteristics for vaccine preparation, like their poor capacity to survive into the gut, their ability to revert to a bile salts resistant phenotype may be advantageous. First, these mutants may prove to be less virulent than the Ty parental strain. Second, considering that the frequency of reversion is variable but generally small, the presence of revertants in the intestinal tract may ensure an efficient immunization. Consequently, such mutants were further grown on TSA supplemented with 500 μg/ml of streptomycin and 9 g/l of bile salts. Bacterial colonies developed with a frequency that was varying from a mutant to another (FIG. 3). These bile salts resistant mutants (BSR) were called Ty An BSR, Ty Bn BSR, Ty Cn BSR, and Ty Vn BSR (where n=1, 2, 3, etc.), like Ty V2 BSR and Ty B63 BSR. Another type of mutant was obtained when SmD mutants were further grown on TSA without streptomycin. Streptomycin-independent revertant (Sm I Rev) colonies developed, like the Ty B63 Sm I Rev (FIG. 3).

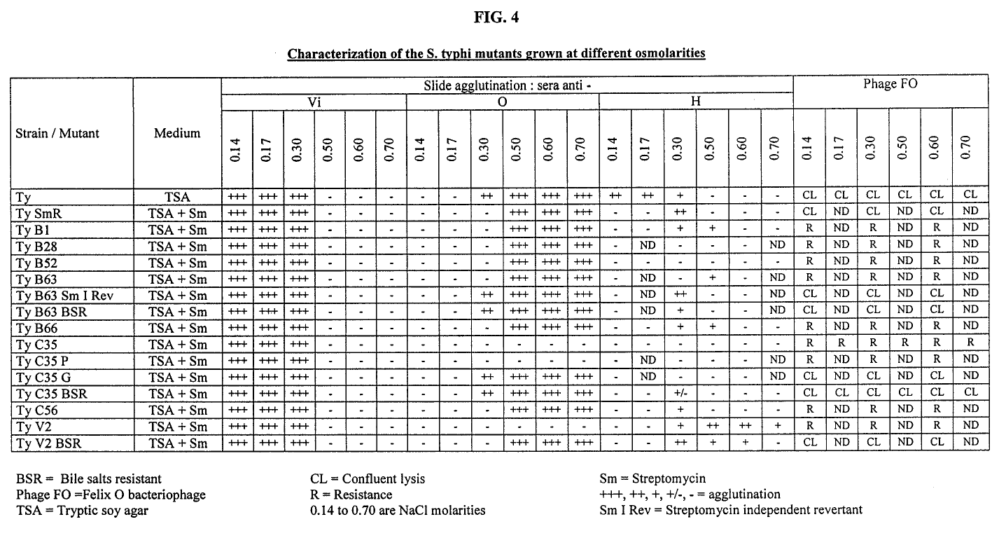

Osmolarity is one, among a number of environmental factors, affecting Salmonella Vi bacterial surface antigen expression (Arricau et al., MOL. MICROBIOL., 29: 835-850 (1998)). To test whether Vi bacterial surface antigen expression in the S. typhi mutants described in the present invention was regulated by osmolarity, we determined the expression of Vi, O, and H antigens in select Ty SmR- and SMD-FOR mutants cultured on media supplemented with sodium chloride concentrations ranging from 0.14 to 0.7 M (FIG. 4). As shown in FIG. 4, the Vi antigen expression in these Salmonella mutant strains is regulated in response to altered osmolarity. Specifically, elevating the NaCl concentration above 0.3 M in the growth media resulted in a complete loss of Vi-surface antigen immunoreactivity even for the mutants that stayed Vi-positive after heating. The concomitant appearance of O surface antigen immunoreactivity at NaCl concentrations greater than 0.3 M in most of the mutants studied is consistent with the fact that the Vi antigen masks the O antigen of the bacterial envelope. However, in a small number of mutants that are Vi-positive after heating, O surface antigen immunoreactivity did not appear, showing that these mutants are rough, like Ty C35, Ty C35 P, and Ty V2 (FIG. 4).

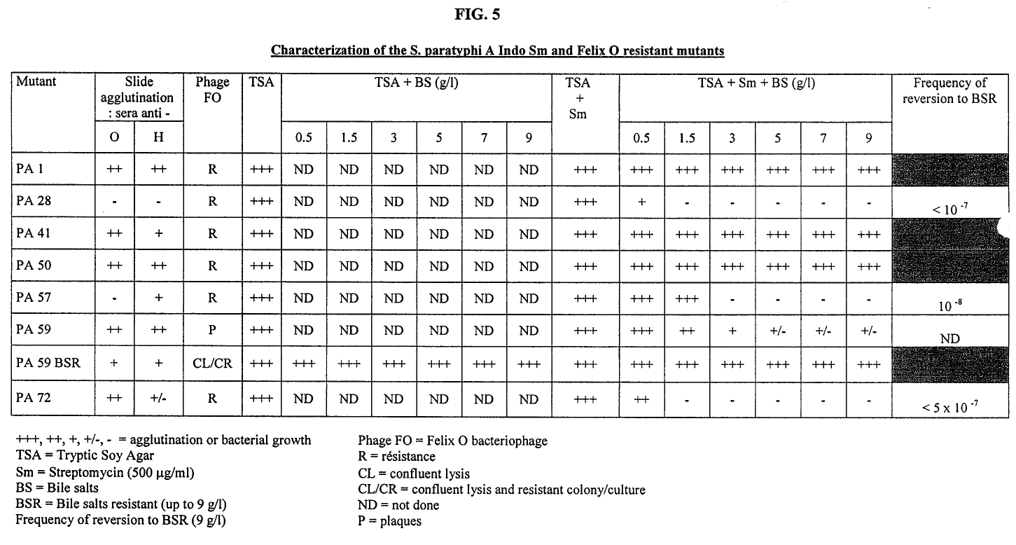

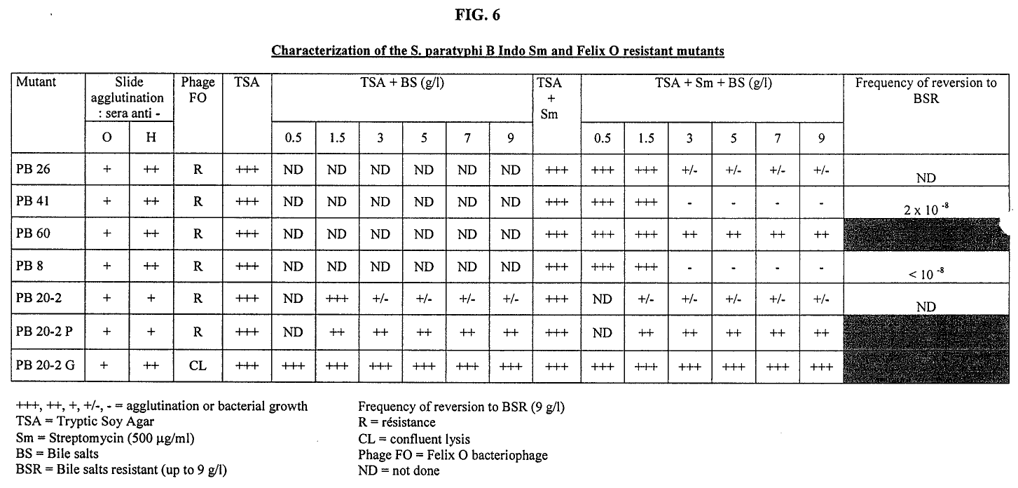

The SmR-FOR mutants derived from PA and PB were characterized by agglutination with specific anti-O and anti-H antisera and by susceptibility to the Felix O bacteriophage (FIGS. 5 and 6). Similarly to the Ty SmR-FOR mutants, the PA- and PB-SmR-FOR mutants were sensitive to different concentrations of bile salts. For example, PA1, PA41, PA50, PB60, and PB20-2 P grew well on media containing 9 g/l of bile salts, whereas PA28, PA57, PA59, PA72, PB26, PB41, PB8, and PB20-2 stopped growing at bile salts concentration lower than 9 g/l (FIGS. 5 and 6). It was shown with Ty SmR-FOR mutants sensitive to bile salts concentration lower than 9 g/l that revertants resistant to bile salts can be obtained at a low frequency (FIG. 3). Similarly, the PA- and PB-SmR-FOR mutants sensitive to low concentration of bile salts can be induced to revert into bile salts resistant mutants by growing them on TSA supplemented with 9 g/l of bile salts. These revertants may be efficient immunogens with attenuated virulence.

It should be noted that the Felix O bacteriophage is a lytic phage (Kallings, ACTA PATH. MICROBIOL. SCAND., 70: 446-454 (1967)). Consequently, it is not expected to integrate into the bacterial genome of the FOR mutants. However, we submitted the FOR mutants to mitomycin C used to isolate stably integrated lysogenic phages (Siddiqui et al., APPL. MICROBIOL., 27: 278-280 (1974)). In addition, FOR mutants and supernatants of FOR mutants cultures have been tested for the presence of Felix O phage by PCR amplification using the primers 5′GCTTCTCCTTCATTGTAG 3′ (SEQ ID NO: 1) and 5′GGGTTCTTACGAGAGTCC 3′ (SEQ ID NO: 2). Both of these procedures confirmed that none of the FOR mutant strains contain integrated Felix O phage.

In contrast with other Salmonella strains, there is no suitable laboratory animal model in which to test the virulence of S. typhi and S. paratyphi mutant strains. Alternatively, in vitro methods such as the in vitro assay of Salmonella survival within human monocyte-derived macrophages (MDM) and the test measuring the susceptibility of Salmonella strains to the bactericidal action of normal human serum have been used to assess the potential virulence of Salmonella mutants. To determine the survival of the SmR- and SmD-FOR mutants in the monocyte-derived macrophages (MDM), we used the human monocytic leukemia cell line THPi-1 (ATCC) induced to differentiate into adherent, macrophage-like cells by treatment for 48 hours (h) with 10⁻⁶ M phorbol-12-myristate-13-acetate (PMA, Sigma) in RPMI 1640 supplemented with 10% (v/v) fetal calf serum (FC S, Gibco-BRL) and 50 μg/ml of gentamicin. Culture of the human monocytic leukemia cell line THP-1 was performed in 96 well plates (Costar), each well containing 6×10⁴ cells. One plate was used to determine the survival of the wild-type strains and mutants after an incubation of 5 h and the other after an incubation of 24 h. Determination of the survival of each of the bacteria for both of the incubation times was based on a mean value obtained from 4 wells.

After 48 h, the medium was drained off and the THP-1 differentiated cells washed once with RPMI 1640. The bacterial suspensions (6×10⁵ bacteria in RPMI 1640 supplemented with 10% (v/v) FCS) were dispensed into each of the wells and the plates incubated for 2 h at 37° C. in an humidified 5% CO₂ atmosphere. The bacterial suspensions were then drained off and the cells washed once with RPMI 1640. The cells were further incubated for 3 h in RPMI 1640 supplemented with 10% (v/v) FCS and 200 μg/ml gentamicin to kill extracellular bacteria. The medium was then drained off, the cells washed twice with RPMI 1640 and lysed with 0.1% Triton X-100 for 20 min at 37° C. in an humidified 5% CO₂ atmosphere or further incubated for 19 h in RPMI 1640 supplemented with 10% (v/v) FCS and 10 μg/ml gentamicin for the determination of the survival after 24 h. The plates were then transferred at room temperature and the content of the wells, kept on ice, was plated on TSA (wild-type strains) and on TSA supplemented with 500 μg/ml of streptomycin (mutants). The number of colony forming units (cfu) was counted and a mean value calculated. The survival of each of the wild-type strains (Ty, PA, PB) after a 5 h incubation time was set to 100% as a reference. The survival of the mutants after a 5 h and a 24 h incubation time and of the wild-type strains after a 24 h incubation time was expressed as a percentage of the reference.

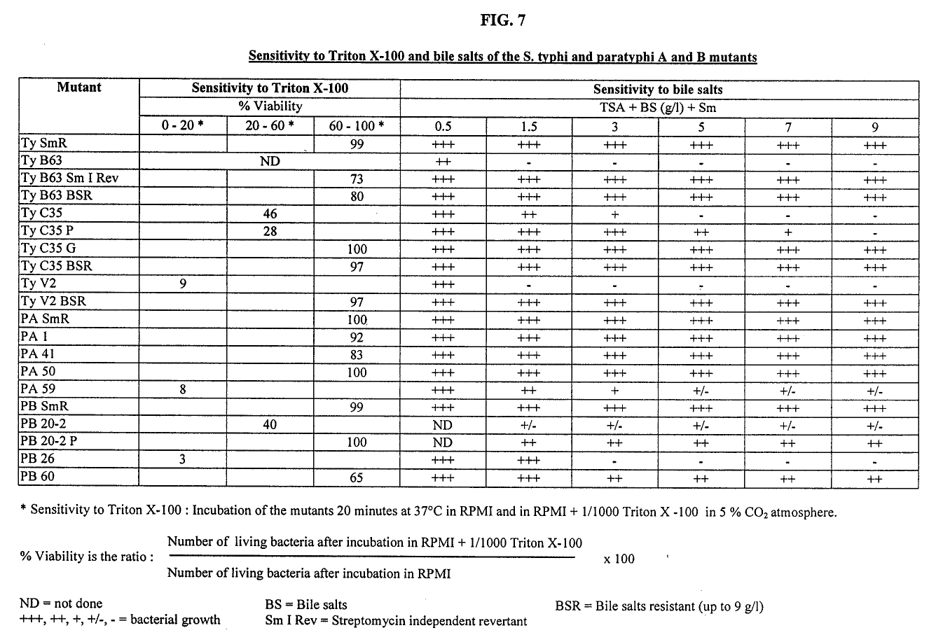

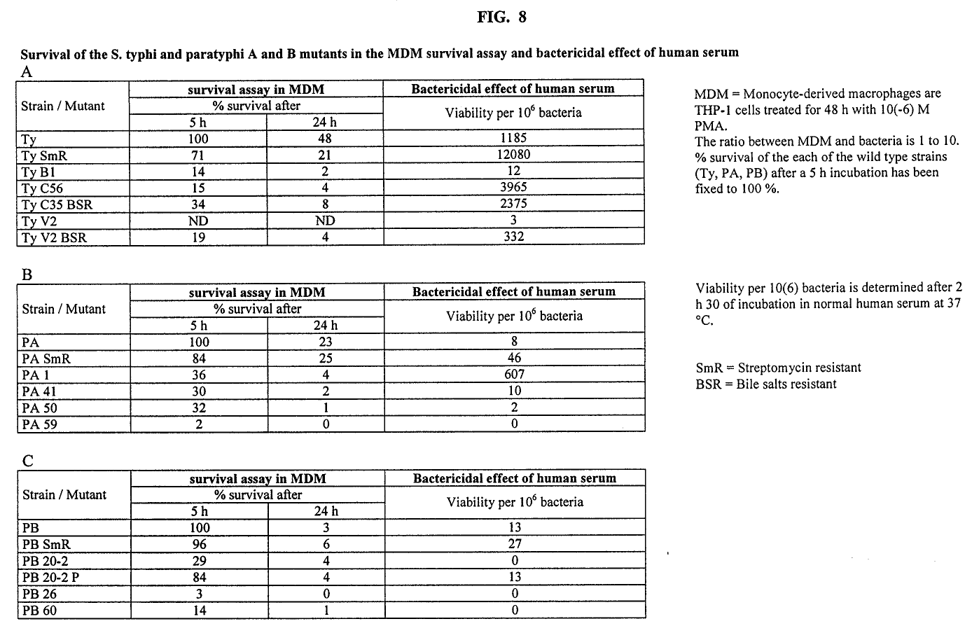

As shown in FIG. 8, SmR- and SMD-FOR Salmonella mutants do not survive as well as the virulent wild-type parental strains in the MDM survival assay, indicating that they are less virulent. For example, Ty B1 and Ty C56 mutants survival rates were approximately 85% lower than the parental Ty strain (100%) after 5 h of incubation. Furthermore, although almost 50% of the parental Ty strain survived for 24 h within the MDM, essentially none of the Ty B I and Ty C56 mutants survived inside the MDM for 24 h. Similar observations were made for other mutants analyzed in FIG. 8. However, it was shown that survival rates of the Salmonella mutants in the MDM assay may be influenced by the sensitivity of the mutants to Triton X-100 used to lyse the MDM prior to plating the bacteria on solid media (FIG. 7). For example, the Ty V2 mutant could not be evaluated in the MDM assay following the present protocol. To determine the sensitivity to Triton X-100 of the mutants derived from Ty, PA, and PB, 100 μl of bacterial suspension (104 bacteria) in RPMI 1640 (Gibco-BRL) and in RPMI 1640+0.1% Triton X-100 were incubated in duplicate in 96 well plates (Costar) for 20 min in a humidified 5% CO₂ atmosphere at 37° C. The plates were then transferred on ice and the content of the wells was plated on TSA supplemented with 500 μg/ml of streptomycin. The number of cfu was counted and a mean value calculated. The sensitivity to Triton X-100 is reported in FIG. 7 as percent viability.

The sensitivity of the Salmonella mutants to the bactericidal action of normal human serum is another mean to evaluate their virulence and an indication if they may generate bacteremia in vaccines. To determine the sensitivity of the mutants, 100 μl of a bacterial suspension (about 10⁶ cfu, estimated by optical density) was mixed in 1.5 ml tubes with 400 μl of normal human serum (25 years old man, serologically negative for hepatitis B, syphilis, HIV, and without a history of typhoid-paratyphoid fevers and not vaccinated against typhoid-paratyphoid) and 100 μl of this mixture was used to count the number of cfu. The tubes were then incubated at 37° C. for 2 h 30 min and then transferred on ice. An aliquot of 100 μl was used to count the number of cfu by plating on TSA or TSA supplemented with 500 μg/ml of streptomycin. The sensitivity to human serum is reported in FIG. 8 as the viability per 106 bacteria.

These data indicated that the PA and PB mutants are generally much more sensitive to human serum than the Ty mutants. Moreover, in each groups of mutants derived from Ty, PA, and PB, several mutants had a reduced survival in MDM, a reduced viability in serum, like Ty B1, Ty V2 BSR, PA50, and PB60, and express the main protective surface antigens. As such, these mutants may be considered potential vaccine candidates. However, the safety and immunogenicity of the S. typhi and S. paratyphi A and B mutants can only be demonstrated when administered to human volunteers.

In a preferred embodiment of the present invention, vaccines against typhoid fever, against paratyphoid A fever, against paratyphoid B fever, and against other diseases caused by Salmonella, comprise:
(a) a pharmaceutically effective preparation containing one or a combination of Salmonella mutants, wherein said mutants are derived from S. typhi, S. paratyphi A, S. paratyphi B, or from another Salmonella strain and are obtained by selection such that it is: (i) resistant or dependent to streptomycin sulphate; (ii) is resistant to Felix O bacteriophage; and (iii) resistant to bile salts; and
(b) a pharmaceutically acceptable carrier or diluent.
   B. Vaccines Against Heterologous Pathogens

In another aspect of the present invention, the live, attenuated Salmonella mutant strains are used as live vector vaccines for delivering foreign antigens to antigen-presenting cells (APC) and eliciting humoral and/or cellular immune responses to the foreign antigens, at the level of both systemic and mucosal compartments.

Bacterial live vectors offer a highly versatile means of delivering protective vaccine antigens with foreign genes under the control of a prokaryotic promoter, preferably an inducible promoter like the Pₙᵢᵣ₁₅ promoter (Chatfield et al., BIO/TECHNOLOGY, 10: 888-892 (1992)) or DNA vaccines with foreign genes under the control of an eukaryotic promoter (Medina et al., VACCINE, 19: 1573-1580 (2001); Dietrich et al., ANTISENSE & NUCLEIC ACID DRUG DEV., 10: 391-399 (2000)). Interestingly, the presence of the plasmids encoding the foreign antigens in the recombinant strains do not necessarily impair the response against the live bacterial vector, encouraging the use of attenuated Salmonella as carriers of multiple antigens, as shown with Fragment C of tetanus toxin expressed in the live attenauted S. typhi CVD 915 (Pasetti et al., CLIN. IMMUNOL., 92: 76-89 (1999)).

Attenuated Salmonella as Live Vectors for Foreign Antigens

The particular Salmonella strains employed as a starting materials in the present invention are not critical thereto. Live attenuated Salmonella strains were transformed by electroporation with plasmids containing genes cloned from foreign pathogens under the control of an inducible or constitutive prokaryotic promoter in 10 or 15% glycerol-water using a Gene Pulser (Bio-Rad) set at 1800 Volts and 400 Ohms. Expression of the foreign antigens was first checked in vitro: following overnight growth on TSA supplemented with 500 μg/ml of streptomycin, bacterial colonies were further grown in TSB supplemented with 500 μg/ml of streptomycin, spinned and resuspended in sterile Phosphate Buffered Saline (PBS) to an OD₆₀₀ of 1.0. Whole-cell bacterial lysates were prepared by harvesting 1 ml aliquots of the suspensions by centrifugation and lysing the pellet in 200 μl of SDS-PAGE sample buffer. Bacterial lysates were separated by electrophoresis through SDS-PAGE gels. Protein bands were visualized by staining with Coomassie brilliant blue or immunoblotting. For immunoblotting, proteins were electrotransferred onto nitrocellulose membranes which were subsequently blocked with 10% skimmed milk in PBS and then incubated at room temperature for 1-2 h with mouse or rabbit polyclonal antibodies against foreign antigen in 1% milk in PBS containing 0.05% Tween 20. Membranes were then incubated with anti-mouse or anti-rabbit immunoglobulins conjugated to horseradish peroxydase (HRP) (Sigma) and reactive polypeptides were visualized using the ECL Plus Western blotting detection reagents (Amersham-Pharmacia).

Transformed live attenuated Salmonella expressing the foreign antigens were then used in vaccine preparations and tested in mice for their immunogenicity and their capacity to induce a protective immune response.

In another embodiment of the present invention, vaccines against pathogens foreign to Salmonella, against typhoid fever, against paratyphoid A fever, against paratyphoid B fever, and against other diseases caused by Salmonella, comprise:
(a) a pharmaceutically effective preparation containing one or a combination of Salmonella mutants, wherein said mutants are derived from S. typhi, S. paratyphi A, S. paratyphi B, or from another Salmonella strain and are obtained by selection such that it is: (i) resistant or dependent to streptomycin sulphate; (ii) is resistant to Felix O bacteriophage; and (iii) resistant to bile salts, and wherein each said mutant encodes and expresses a foreign antigen under the control of a prokaryotic promoter (i.e., bacterial expression of the foreign antigen); and
(b) a pharmaceutically acceptable carrier or diluent.

The particular foreign antigen employed in the Salmonella live vector is not critical to the present invention. The attenuated Salmonella strains of the present invention may be used as live vectors for immunization against enteric pathogens (pathogen defined as bacterial, viral, etc.), sexually transmitted disease pathogens, acute respiratory tract disease pathogens or pathogens with a mucosal entry that lead to grave systemic manifestations of disease (e.g., meningococcal disease). It may also be used to protect against different types of parasitic infections, such as Plasmodium falciparum (P. falciparum), Leishmania species, Entameba histolytica (E. histolytica) and Cryptosporidium.

Examples of antigens from enteric pathogens that may be expressed in the Salmonella live vectors of the present invention include:
(a) Fimbrial colonization factors of enterotoxigenic Escherichia coli (E. coli), and either the B subunit or heat-labile enterotoxin or a mutant heat-labile enterotoxin (that does not cause intestinal secretion but, elicits neutralizing antitoxin);
(b) Fimbrial antigens and/or intimin of enterohemorrhagic E. coli, along with B subunit of Shiga-toxin 1 or 2 (or mutant Shiga toxin 1 or 2);
(c) O antigens of Shigella and invasion plasmid antigens or VirG of Shigella;
(d) Neutralization antigens from rotaviruses;
(e) Urease, colonization antigens and other protective antigens from H. pylori; and
(f) Inactivated Clostridium difficile toxins or antigens derived from them.

Examples of antigens from sexually-transmitted pathogens that may be expressed in the Salmonella live vectors of the present invention include:
(a) Pili and outer membrane proteins of Neiserria gonorrhoae; and
(b) Proteins of Chlamydia trachomatis.

Examples of antigens from acute respiratory tract pathogens that may be expressed in the Salmonella live vectors of the present invention include:
(a) A mutant diphtheria toxin with substitutions in the NAD binding domain that lacks toxic activity yet, elicits neutralizing antitoxin;
(b) Antigens of Bordetella pertussis, including a fusion protein consisting of the truncated S1 subunit of pertussis toxin fused to fragment C of tetanus toxin, mutant pertussis toxin, filamentous hemagglutinin and pertactin;
(c) F and G glycoproteins of Respiratory Syncytial Virus;
(d) Capsular polysaccharide of Haemophilus influenzae type b; and
(e) Capsular polysaccharides of group A and C Neisseria meningitidis, and outer membrane proteins of group B Neisseria meningitidis.

Examples of antigens from parasites that may be expressed in the Salmonella live vectors of the present invention include:
(a) The circumsporozoite protein (CSP), Liver Stage Antigen-1 (LSA-1), SSP-2 (also known as TRAP) and Exp-1 of P. falciparum;
(b) Asexual erythrocytic stage antigens of P. falciparum, including MSP-1, MSP-2, SERA, AMA-1;
(c) Sexual stage antigens of P. falciparum, including Pfs25, and gp63 of Leishmania species; and
(d) The serine-rich E. histolytica protein (SREHP).
   Attenuated Salmonella as Live Vectors for Foreign DNAs

Expression of the foreign antigens was first checked in vitro: the CHO dhfr⁻ cells (CHO DUK⁻) were obtained from the American Type Culture Collection (ATCC CRL 9096) and transfected by electroporation with the plasmids containing the genes cloned from the foreign pathogens. CHO dhfr⁻ cells were cultured in α-minimal essential medium (MEM-α) supplemented with 10% dialyzed fetal calf serum (FCS), 10 mM Hepes (pH 7.0), and 50 μg/ml gentamycin. In the mid- to late-logarithmic phase of growth, the cells were released from plastic by trypsin-EDTA. Cells (2×10⁶) were electroporated (400 V; 250 μF; 2 pulses at an interval of 1 nm) with 30 μg of linearized plasmid in Hanks' balanced salt solution (HBSS) supplemented with 20 mM Hepes (pH 7.0), 0.108% glucose, and 0.5% FCS. Transfected cells were then transferred in a 35-mm culture dish containing fresh growth medium and incubated at 37° C. in an humidified 5% CO₂ atmosphere. The cells were harvested 24 to 96 hours after transfection and the resulting lysates or cell culture supernatants were assayed for expression of the target genes by ELISA or by immunoblotting.

Live attenuated Salmonella strains were then transformed by electroporation with plasmids containing genes cloned from foreign pathogens under the control of an inducible or constitutive eukaryotic promoter in 10 or 15% glycerol-water using a Gene Pulser (Bio-Rad) set at 1800 Volts and 400 Ohms.

The particular Salmonella strains employed as a starting materials in the present invention are not critical thereto.

Transformed live attenuated Salmonella expressing the foreign antigens were then used in vaccine preparations and tested in mice for their immunogenicity and their capacity to induce a protective immune response.

In yet another embodiment of the present invention, vaccines against pathogens foreign to Salmonella, against typhoid fever, against paratyphoid A fever, against paratyphoid B fever, and against other diseases caused by Salmonella, comprise:
(a) a pharmaceutically effective preparation containing one or a combination of Salmonella mutants, wherein said mutants are derived from S. typhi, S. paratyphi A, S. paratyphi B, or from another Salmonella strain and are modified by selection such that it is; (i) resistant or dependent to streptomycin sulphate; (ii) is resistant to Felix O bacteriophage; and (iii) is resistant to bile salts, and wherein each said mutant contains a plasmid which encodes and expresses, using an eukaryotic promoter, in an eukaryotic cell, a foreign antigen; and
(b) a pharmaceutically acceptable carrier or diluent.

The particular foreign antigen employed in the DNA-mediated vaccine is not critical to the present invention. Examples of such antigens include those from a variety of pathogens, such as influenza (Justewicz et al., J. VIROL. 69: 7712-7717 (1995); Fynan et al., INT. J. IMMUNOPHARMACOL. 17: 79-83 (1995)), lymphocytic choriomeningitis virus (Zarozinski et al., J. IMMUNOL. 154: 4010-4017 (1995)), human immunodeficiency virus (Shiver et al., ANN. NY. ACAD. SCI., 772: 198-208 (1995)), hepatitis B virus (Davis et al., VACCINE, 12: 1503-1509 (1994)), hepatitis C virus (Lagging et al., J. VIROL. 69: 5859-5863 (1995)), rabies virus (Xiang et al., VIROLOGY 209: 569-579 (1995)), Schistosoma (Yang et al., BIOCHEM. BIOPHYS. RES. COMMUN. 212: 1029-1039 (1995)), Plasmodium (Sedegah et al., PROC. NATL. ACAD. SCI., USA, 91: 9866-9870 (1994)); and mycoplasma (Barry et al, NATURE 377: 632-635 (1995)).

Immunization of mice with live attenuated Salmonella vectors that express antigens foreign to Salmonella or deliver DNA vaccines were carried as follows:

Serologic and cellular immune responses against Salmonella antigens and foreign antigens were measured following nasal (mucosal) immunization of mice. Following overnight culture at 37° C., vaccine strains were harvested from TSA plates supplemented with 500 μg/ml of streptomycin and resuspended in 10 ml of sterile PBS. The bacterial suspensions were diluted to an optical density at 600 nm of 0.5 (equivalent to 5×10⁸ cfu/ml) and concentrated to 1×10¹¹ cfu/ml by centrifugation and resuspension in an appropriate volume of sterile PBS. Balb/c mice were immunized intranasally (i.n.) with approximately 2×10⁹ cfu of attenuated recombinant Salmonella in a 30 μl volume. Mice were boosted in an identical manner 35 days later. Control mice received PBS i.n.

Humoral and cellular immune responses were assayed as follows:

Mice were bled and sera stored at −70° C. until tested. Total IgG antibodies and IgG subclasses against the foreign antigens, and Salmonella antigens were determined by ELISA. Briefly, 96 well plates were coated with 100 μl of purified foreign antigens, or Salmonella antigens during 3 h at 37° C. and blocked overnight with 10% milk in PBS. Plates were washed five times with PBS containing 0.05% Tween 20 (PBST) after each incubation. Eight twofold dilutions of each sera in 10% PBST were incubated for 1 h at 37° C. Peroxidase conjugates anti-IgG; anti-IgG1, -IgG2a, -IgG2b, and -IgG3 (Roche) were diluted 1/1000 in the same diluent and incubated for 1 h at 37° C. The substrate solution used was o-phenylenediamine (1 mg/ml) and H₂O₂ (0.03%; Sigma) in 0.1 M phosphate citrate buffer (pH 5). After a 15 nm incubation, the reaction was stopped by the addition of 2 M H₂SO₄ and the optical densities at 492 nm were measured in an ELISA microplate reader (Labsystems Multiskan MS). Tests and controls were run in duplicates. Linear regression curves were plotted for each serum to calculate antibody titers.

Cervical lymph nodes, mesenteric lymph nodes, and spleens were taken from five animals in each group and pooled. Single cell suspensions were prepared and resuspended in RPMI 1640 supplemented with 2 mM L-glutamine, 10 mM Hepes, 50 μg/ml gentamicin, and 10% heat-inativated fetal calf serum (Gibco-BRL). Antigen-specific proliferative responses were measured by culturing 2×10⁵ cells/well (triplicate wells) in 96-well round bottom plates with purified foreign antigens, Salmonella antigens, or Bovine serum albumin (BSA). Whole-cell heat phenolyzed Salmonella were added at 2×10⁵ and 2×10⁴ particles/well. The final volume was always 200 μl. Cells were cultured for 6 days at 37° C. under 5% CO₂. As a control, each cell population was cultured with 2 μg/ml PHA under the same conditions and harvested 3 days later. Cultures were pulsed with 1 μCi/well of tritiated thymidine and harvested 18-20 h later. Cellular proliferation was measured by incorporation of [³H]thymidine, measured in a Wallac Microbeta counter.

The decision whether to express the foreign antigen in Salmonella (using a prokaryotic promoter in a live vector vaccine) or in the cells invaded by Salmonella (using a eukaryotic promoter in a DNA-mediated vaccine) may be based upon which vaccine construction for that particular antigen gives the best immune response in animal studies or in clinical trials, and/or, if the glycosylation of an antigen is essential for its protective immunogenicity, and/or, if the correct tertiary conformation of an antigen is achieved better with one form of expression than the other.

IV. Vaccine Formulation

In the vaccines of the present invention, the pharmaceutically effective amount of the mutants of the present invention to be administered may vary depending on the age, weight and sex of the subject, and the mode of administration. Generally, the dosage employed will be about 10² cfU to 10¹⁰ cfu. Preferably, about 10⁶ cfu to 10¹⁰ cfu is used for an oral administration in which vaccine is given in capsules or suspended in a buffer solution to protect the attenuated bacteria against acidic pH in the stomach; or about 10² cfu to 10⁷ cfu is used for intranasal administration in which the bacteria is given in drops or aerosol.

The particular pharmaceutically acceptable carrier or diluent employed is not critical to the present invention, and are conventional in the art. Examples of diluents include: buffers for buffering against gastric acid in the stomach, such as citrate buffer (pH 7.0) containing sucrose, bicarbonate buffer (pH 7.0) alone (Levine et al., REV. INFECT. DIS. 11 (supp 3): S552-S567 (1987); Black et al, VACCINE 8: 81-84 (1990)), or bicarbonate buffer (pH 7.0) containing ascorbic acid, lactose, and optionally aspartame (Levine et al., LANCET II: 467-470 (1988)). Examples of carriers include: proteins, e.g., as found in skim milk; sugars, e.g., sucrose; or polyvinylpyrrolidone. The mutants of the present invention can be stored at −80° C. while suspended in TSB (Difco) containing 15% (v/v) glycerol and 500 μg/ml of streptomycin.

V. Strain Deposit

Under the terms of the Budapest Treaty on the International Recognition of the Deposit of Microorganisms for the Purpose of Patent Procedure, deposit of the following materials has been made with the American Type Culture Collection (ATCC) of Manassas, Va., USA.

Applicant's assignee, the Galli Valerio Foundation, represents that the ATCC is a depository affording permanence of the deposit and ready accessibility thereto by the public if a patent is granted. All restrictions on the availability to the public of the material so deposited will be irrevocably removed upon the granting of a patent. The material will be available during the pendency of the patent application to one determined by the Commissioner to be entitled thereto under 37 CFR 1.14 and 35 U.S.C. § 122. The deposited material will be maintained with all the care necessary to keep it viable and uncontaminated for a period of at least five years after the most recent request for the furnishing of a sample of the deposited plasmid, and in any case, for a period of at least thirty (30) years after the date of deposit or for the enforceable life of the patent, whichever period is longer. Applicants' assignee acknowledges its duty to replace the deposit should the depository be unable to furnish a sample when requested due to the condition of the deposit.

Salmonella typhi mutant strain TyB 1 has been deposited with the American Type Culture Collection (Manassas, Va.) and has received ATCC designation PTA-3733.

Salmonella paratyphi A mutant strain PA50 has been deposited with the American Type Culture Collection (Manassas, Va.) and has received ATCC designation PTA-3734.

Salmonella paratyphi B mutant strain PB60 has been deposited with the American Type Culture Collection (Manassas, Va.) and has received ATCC designation PTA-3735.

The following examples are provided for illustrative purposes only, and are in no way intended to limit the scope of the present invention.

### EXAMPLE 1

### Characterization of the Live Mutants

1. Streptomycin-Resistant Mutants

Mutants of S. typhimurium, resistant or dependent to streptomycin were shown to accumulate mutations in the 16 S ribosomal RNA and in several ribosomal proteins (Allen et al., CELL, 66: 141-148 (1991); Bjorkman et al., PROC. NATL. ACAD. USA, 95: 3949-3953 (1998)).

Similar studies conducted on other bacterial strains like Mycobacterium tuberculosis showed that accumulation of mutations in the 16 S ribosomal RNA and in the ribosomal proteins is dependent on the level of resistance to streptomycin. Streptomycin-highly resistant mutants and mutants resistant to low concentration of streptomycin do not accumulate the same mutations (Katsukawa et al., J. APPL. MICROBIOL., 83: 634-640 (1997)).

Based on these observations, streptomycin-resistant (SmR) mutants have been obtained from the parental strains S. typhi, S. paratyphi A and S. paratyphi B in a single step of selection in the presence of a high concentration of streptomycin (500 μg/ml). One single SmR mutant was isolated for each of the S. typhi, S. paratyphi A and S. paratyphi B which was shown to be less virulent than the wild-type strain using the monocyte-derived macrophage (MDM) survival assay (FIG. 8). These mutants, named Ty SmR, PA SmR, and PB SmR were further characterized by slide agglutination with sera anti-O, anti-Vi (only for the Ty SmR) and anti-H. They do not significantly differ from the wild-type strains for these antigens and for their growth on solid media supplemented with bile salts up to a concentration of 9 g/l (FIG. 2).

2. STREPTOMYCIN and Felix O Bacteriophage-Resistant Mutants

Different bacteriophages have been described that attach to different regions of the lipopolysaccharide (LPS) molecule: the smooth-specific phages (for example P22, and P27) attach to the O-antigenic side chain, the rough-specific phages (for example 6SR, Br2, and Br60) attach to the core and the phages that attach to both the smooth and rough forms of the LPS, like the Felix O (Felix O-1, also called FO phage) (MICROBIAL TOXINS, Ajl et al. ed., NY Academic 1971; Lindberg et al., J. BACTERIOL., 105: 57-64 (1971)). In general, the smooth-specific phages are all temperate converting phages that alter the structure of the O-antigen whereas the rough phages are all virulent, although there may be exceptions.

Felix O resistant (FOR) mutants are resistant to the virulent Felix O bacteriophage, whose receptor includes the N-acetylglucosamine branch of the LPS core (Felix et al., BRIT. MED. J., 2: 127-130 (1943); Lindberg et al., J. BACTERIOL., 99: 513-519 (1969); MacLachlan et al., J. BACTERIOL., 173: 7151-7163 (1991); Heinrichs et al., J. BIOL. CHEM., 273: 8849-8859 (1998)). Smooth strains whose LPS core bears O chain and rough strains, which make complete core LPS without O chains, are lysed by phage FO. Accordingly, mutants that do not synthesize a complete core are resistant to phage FO and selection of FOR mutants from the streptomycin-resistant typhi and paratyphi A and B consists in the isolation of SmR mutants that have a defective LPS core structure. These mutants may not express active sugar transferases or may not produce the substrates of these enzymes. In either cases, however, other biosynthetic pathways may be affected resulting in mutants with a wide range of different phenotypes.

Four independent rounds of selection of FOR mutants from the Ty SmR strain generated at least 80 mutants representative of more than 1000 mutants obtained originally. These mutants were named Ty Aₙ, Ty Bₙ, Ty Cₙ and Ty Vₙ (where n=1, 2, 3, etc.) (FIG. 1). A representative sample of these mutants was further characterized by slide agglutination with sera anti-O, anti-Vi and anti-H (FIG. 3). Agglutinations were performed before and after a 10 min incubation at 100° C. (boiling water) to characterize the O antigen of the LPS which is masked by the Vi antigen before heating. Surprisingly, two classes of mutants resulted from the anti-O agglutination. The first one consists of mutants that stayed negative or slightly positive for the O antigen after heating and positive for the Vi antigen (FIG. 3). This property has not been reported for other Salmonella mutants. The second one consists of mutants with regular features, that become positive for the O antigen after heating and negative for the Vi antigen (FIG. 3).

One round of selection of FOR mutants was performed on both PA SmR and PB SmR strains. Seventeen PA SmR-FOR mutants and 18 PB SmR-FOR mutants were representative of more than 140 PA- and 60 PB-derived mutants obtained originally. These mutants were named PAₙ and PBₙ (where n=1, 2, 3, etc.) (FIG. 1). The representative PA and PB mutants were further characterized by slide agglutination with sera anti-O and anti-H. Results are reported in FIGS. 5 and 6. Growth of these SmR-FOR mutants on media containing bile salts (0.5 to 9.0 g/l) showed that some mutants grow well up to 9.0 g/l of bile salts and some stopped growing at 0.5, 1.5, 3.0, 5.0 or 7.0 g/l of bile salts. This behavior was observed for the Ty, PA, and PB-derived mutants (FIGS. 3, 5, 6). In addition, both classes of the Ty SmR-FOR mutants that are either Vi-positive or Vi-negative after heating grow well either up to 9.0 g/l of bile salts or up to a lower concentration, suggesting that both Vi-anchor in the membrane and growth on media containing bile salts are under the control of independent regulatory elements. In addition, it was clearly established that Vi expression is regulated by osmolarity in both classes of Ty SmR-FOR mutants (FIG. 4). Moreover, growth of the mutants that were Vi-positive after heating on media with increasing osmolarities was also a way to determine whether the mutants are originally O-negative (C35, C35 P, and V2 which are rough) or O-positive with an O antigen masked by the Vi (B1, B28, B63, etc. which are smooth). This experiment also showed that the H antigen (flagellin) is regulated by osmolarity with a maximum of expression around 0.3 M NaCl (FIG. 4).

3. Streptomycin-Dependent and Felix O Bacteriophage-Resistant Mutants

Several streptomycin-dependent (SmD) mutants, for example Ty V2 and Ty B63 (FIG. 3), were also obtained when selection of FOR mutants from the Ty SmR strain was performed. These mutants only grow in the presence of streptomycin and result from mutations in the 16 S ribosomal RNA and in ribosomal proteins of the 30 S subunit that differ from those of the SmR mutants. Ty V2 and Ty B63 are both Vi-positive after heating and grow well only on media containing 0.5 g/l of bile salts. Moreover, Ty V2 was shown to be resistant to the FO phage and Ty B63 only partially resistant.

4. Streptomycin-Independent Revertant Mutants

Streptomycin-independent revertant (Sm I Rev) mutants, for example Ty B63 Sm I Rev (FIG. 3) are obtained when large amount of Ty B63 SmD mutant are plated on a medium that do not contain streptomycin. Such mutant accumulate mutations in the 16 S ribosomal RNA and in ribosomal proteins of the 30 S subunit that differ from the SmD mutation that usually persists. Such Sm I Rev mutants can grow either in absence or in presence of streptomycin. The Ty B63 Sm I Rev was shown to loose its resistance to the FO phage, to become mostly O-positive after heating and to grow well on media containing bile salts in a concentration as high as 9 g/l. In fact, the Ty B63 Sm I Rev has properties that are very close to that of Ty B63 BSR and of the original Ty SmR mutant.

5. Streptomycin- and Bile Salts-Resistant Mutants

Streptomycin- and bile salts-resistant (BSR) mutants, for example Ty V2 BSR, Ty B63 BSR and Ty C35 BSR (FIG. 3), are obtained when large amount of Ty V2, Ty B63 and Ty C35, which are all resistant to low concentrations of bile salts, are plated on a medium containing 9 g/l of bile salts. Such mutants were shown to loose resistance to the FO phage and to become Vi-negative after heating suggesting that synthesis of the FO phage receptor (LPS) and anchor of the Vi antigen in the bacterial membrane are under common regulatory elements. The frequency of reversion to bile salts resistance (on a medium containing 9 g/l of bile salts) was quite variable, ranging from 1 revertant over 10,000 (Ty C35) to 1 revertant over 10 millions (Ty V2).

### EXAMPLE 2

### Virulence of the Mutants

1. In Vitro Evaluation Using the Human Monocyte-Derived Macrophage Survival Assay and the Bactericidal Effect of Human Serum

When evaluating new attenuated S. typhi, S. paratyphi A and B mutants as possible live vaccines, reliable and representative in vitro assays are needed. Indeed, lack of an appropriate animal model that reproduces human typhoid/paratyphoid infection limits the meaningful evaluation of such attenuated Salmonella strains. However, evaluation of virulence of attenuated strains of S. typhimurium can be performed in mice because mice are naturally susceptible to S. typhimurium and develop an enteric-type illness bearing a resemblance to typhoid fever. Accordingly, two in vitro tests are commonly used to assess virulence of such mutants. The first one is the monocyte-derived macrophage (MDM) survival assay (Vladoianu et al, MICROB. PATHOG., 8: 83-90 (1990); Sizemore et al., INFECT. IMMUN., 65: 309-312 (1997)) and the second one is the bactericidal effect of the human serum (Joiner, CURR. TOPICS MICROBIOL. IMMUNOL., 121: 99-133 (1985).

The MDM survival assay was shown to reflect the ability of Salmonella to survive within macrophages after passage through the intestinal mucosa. This ability to survive within macrophages constitutes an essential step in the pathogenesis that is influenced by bacterial virulence and host-dependent factors. In particular, evaluation of the virulence of wild-type and attenuated S. typhimurium strains with the MDM assay performed with mouse macrophages correlated with evaluation of their virulence in mice (Buchmeier et al., INFECT. IMMUN., 57: 1-7 (1989)). However, virulent S. typhi Ty2 was killed in mouse macrophages but was able to survive within human MDM (Vladoianu et al., MICROB. PATHOG., 8: 83-90 (1990)). Consequently, the evaluation of virulence of the S. typhi, S. paratyphi A and B derived mutants in the MDM survival assay requires human macrophage-like cells. After differentiation into adherent macrophage-like cells, primary monocytes from healthy volunteers, the human myelomonocytic U937 cell line and the human monocytic leukemia cell line THP-1 have proven useful in the MDM survival assay (Sizemore et al., INFECT. IMMUN., 65: 309-312 (1997); Dragunsky et al., VACCINE, 8: 263-268 (1990); Hirose et al., FEMS MICROBIOL. LETT., 147: 259-265 (1997)). To better standardize the assay, we have chosen to use the THP-1 cell line induced to differentiate for 48 h in the presence of 10⁻⁶ M phorbol-12-myristate-13-acetate (PMA) (Schwende et al, J. LEUK. BIOL., 59: 555-561 (1996)).

FIG. 8 shows that the SmR mutants derived from Ty, PA, and PB are only slightly attenuated and even that PB SmR is not significantly different from PB in this assay. However, the corresponding SmR-FOR mutants were shown to be highly attenuated in comparison with the SmR mutants and the degree of attenuation ranged from almost avirulence to intermediate virulence and to higher virulence. Data collected after 5 h (2 h of incubation of the THP-1 differentiated cells with the bacterial suspension followed by 3 h of incubation with a high concentration of gentamicin to kill the extra-cellular bacteria) mostly reflect the capacity of the mutants to penetrate the macrophages and data collected after 24 h (2 h of incubation of the THP-1 differentiated cells with the bacterial suspension followed by 3 h of incubation with a high concentration of gentamicin to kill the extra-cellular bacteria and 19 h of incubation with a low concentration of gentamicin) mostly reflect the capacity of the mutants to survive within the macrophages.

It results from the data in FIG. 8 that some SmR-FOR mutants invade the macrophages more efficiently (these are the mutants with the highest % survival after 5 h: Ty C35 BSR, PA1, PB 20-2 P) and others better survive within the macrophages (these are the mutants with the lowest reduction of % survival between 5 and 24 h: Ty C56, PA 1, PB 20-2). In addition, these data demonstrated that the MDM survival assay performed with the THP-1 differentiated cells is useful for the selection of possible vaccine candidates.

The bactericidal effect of the human serum on enteric gram-negative organisms is, in addition, an indirect evaluation of their virulence. Indeed, lipopolysaccharide (LPS) from these bacteria display extensive size heterogeneity and activate the alternative pathway of complement to different extends (Grossman et al., J. BACTERIOL., 169: 856-863 (1987)). Smooth forms of the Salmonella are generally relatively virulent and resistant to the bactericidal action of normal serum whereas rough forms are avirulent and more susceptible.

Another component of the bacterial membrane, the Vi antigen, was shown to play a role in the sensitivity to human serum of S. typhi and the mutants derived thereof but not of S. paratyphi A and B which are Vi-negative. The presence of Vi has been correlated, in vitro, with a significant decrease in lysis by serum, complement activation and phagocytosis (Looney et al, J. LAB. CLIN. MED., 108: 506-516 (1986)). Thus, the Vi antigen may act as a shield protecting S. typhi against the immune system. This observation was confirmed by several Vi-negative Ty SmR-FOR mutants which were totally lysed by the serum (data not shown).

Bactericidal effect of human serum on the Ty, PA, and PB strains showed that the Ty and Ty-derived strains are much more resistant to normal human serum than the PA and PB strains with the exception of Ty B1 which is less resistant than the other Ty-derived mutants (FIG. 8). Differences between the mutants can be as big as 1000 times suggesting that some mutants are much more appropriate than others as vaccine strains. Indeed, even silent bacteremia in vaccinated people is not acceptable because immuno-compromized individuals found in large groups of population may be severely affected.

2. In Vivo Evaluation of a Ty SmD-FOR Mutant

The Ty V2 mutant was selected for an in vivo trial on a human volunteer (a 65 years old man, serologically negative for HIV, hepatitis B and without a history of typhoid). Indeed, Ty V2 was shown to be highly sensitive to the bactericidal effect of human serum (FIG. 8) and not likely to induce bacteremia.

This mutant is streptomycin-dependent, resistant to the FO phage, O-negative, Vi-positive and remains Vi-positive after heating, grows on bile salts up to a concentration of 0.5 g/l (FIGS. 3 and 4). In addition, Ty V2 was shown to revert to bile salts resistance with a frequency of 1 out of 10 millions (10⁻⁷) into Ty V2 BSR which becomes streptomycin-independent, sensitive to the FO phage, Vi- and O-positive and grows on bile salts up to a concentration of 9 g/l. In contrast to Ty V2, Ty V2 BSR is not sensitive to Triton X-100 (FIG. 7) and the MDM survival assay showed that this mutant is much less virulent than Ty and Ty SmR (FIG. 8). Consequently, the Ty V2 BSR mutant has potentially a better capacity to survive into the gut and to induce a potent immune response.

Ty V2 was administered in three oral doses containing 5.2×10⁹, 1.7×10¹⁰, and 2.8×10¹⁰ bacteria at intervals of 48 h. The live bacteria were in suspension in 30 ml of milk and swallowed after neutralization of gastric acidity with sodium bicarbonate 5 min before ingestion. Complete fasting was observed 90 min before and after the oral administration of the live bacteria. During one month, starting immediately after the first dose, axillary temperature of the volunteer was measured twice a day and stool culture regularly checked for the presence of Ty V2 and Ty V2 BSR. Temperature stayed around 36° C. and none of the mutants were found in the cultures. Moreover, no digestive troubles appeared and no other particular side effect was observed. Consequently, Ty V2 is considered to be safe at the doses administered in this trial.

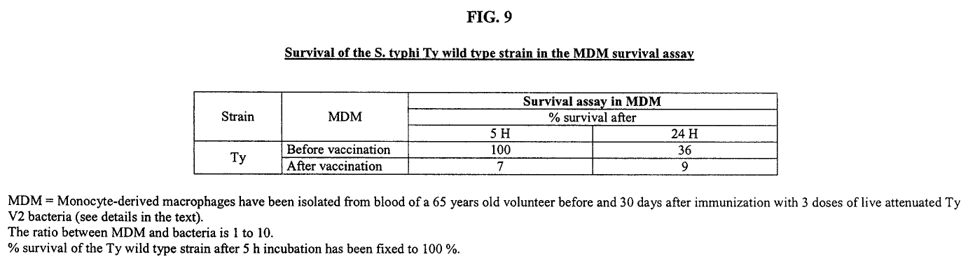

Survival of the Ty wild-type strain was then tested in the MDM assay with monocyte-derived macrophages from the volunteer prepared before and 30 days after immunization with the Ty V2 mutant (FIG. 9). Results showed that survival of Ty was greatly decreased after immunization suggesting an efficient induction of immune response.

### Equivalents

From the foregoing detailed description of the specific embodiments of the invention, it should be apparent that a unique procedure to obtain and select live attenuated mutant strains of Salmonella has been described resulting in unique live attenuated mutant strains of S. typhi, S. paratyphi A and S. paratyphi B as matter of example. Although particular embodiments have been disclosed herein in detail, this has been done by way of example for purposes of illustration only, and is not intended to be limiting with respect to the scope of the appended claims which follows. In particular, it is contemplated by the inventor that substitutions, alterations, and modifications may be made to the invention without departing from the spirit and scope of the invention as defined by the claims. For instance, the choice of a particular Salmonella strain to which the procedure of attenuation is applied, or the choice of a particular live attenuated strain of Salmonella as vector for foreign antigen or foreign polynucleotide sequence, or the choice of a particular antigen or of a polynucleotide sequence from a pathogenic organism is believed to be matter of routine for a person of ordinary skill in the art with knowledge of the embodiments described herein.

We claim:

1. A live, attenuated Salmonella mutant of a pathogenic Salmonella strain selected from the group consisting of at least one of a Salmonella typhi mutant, a Salmonella paratyphi A mutant, a Salmonella paratyphi B mutant, and a combination thereof, wherein said attenuated Salmonella mutant has the following characteristics:
(i) resistance or dependence to an antibiotic,
(ii) resistance to a virulent bacteriophage, and (iii) resistance to bile salts,
and wherein the live, attenuated Salmonella mutant induces humoral and/or cellular immunity against Salmonella infection by a pathogenic Salmonella strain selected from the group consisting of at least one of Salmonella typhi, Salmonella paratyphi A and Salmonella paratyphi B.

2. The live, attenuated Salmonella mutant of claim 1, wherein said antibiotic is streptomycin.

3. The live, attenuated Salmonella mutant of claim 1, wherein said bacteriophage is Felix O.

4. The live, attenuated Salmonella mutant of claim 1, wherein said bile salts are cholic and deoxycholic acids.

5. The live, attenuated bacterial mutant of claim 1 wherein said bacteriophage binds to a bacterial virulence factor.

6. The live, attenuated bacterial mutant of claim 1, wherein said attenuated mutant does not revert to its original virulence when administered in a pharmaceutically effective dosage to an host susceptible to said pathogenic bacterial strain.

7. The attenuated Salmonella mutant of claim 1, wherein said attenuated Salmonella typhi mutant is Ty B1 (ATCC No PTA-3733).

8. The attenuated Salmonella mutant of claim 1, wherein said attenuated Salmonella paratyphi A mutant is PA 50 (ATCC No PTA-3734).

9. The attenuated Salmonella mutant of claim 1, wherein said attenuated Salmonella paratyphi B mutant is PB 60 (ATCC No PTA-3735).

10. The attenuated Salmonella typhi mutant of claim 1, wherein said attenuated Salmonella typhi mutant expresses a heat-stable anchoring of the Vi antigen.

11. The live, attenuated bacterial mutant of claim 1, wherein said mutant encodes and expresses a foreign antigen.

12. The live, attenuated bacterial mutant of claim 1, wherein said mutant contains a plasmid which encodes and expresses, in a eukaryotic cell, a foreign antigen.

13. A pharmaceutical composition comprising the live, attenuated mutant of claim 1, and a pharmaceutically acceptable carrier.
